(19) United States
(12) Patent Application Publication
(10) Pub. No.: US 2015/0038856 A1
(43) Pub. Date: Feb. 5, 2015

(54) METHOD AND APPARATUS FOR ESTIMATING MYOCARDIAL CONTRACTILITY USING PRECORDIAL VIBRATION

(71) Applicants: Geoffrey Houlton, Vancouver (CA); Graeme Jahns, Burnaby (CA); Kouhyar Tavakolian, Burnaby (CA)

(72) Inventors: Geoffrey Houlton, Vancouver (CA); Graeme Jahns, Burnaby (CA); Gonzalo Portacio, Toronto (CA); Kouhyar Tavakolian, Burnaby (CA)

(73) Assignee: HEART FORCE MEDICAL INC, VANCOUVER (CA)

(21) Appl. No.: 14/115,583

(22) PCT Filed: May 2, 2012
(86) PCT No.: PCT/CA2012/050282

§ 371 Date: Feb. 25, 2014

Related U.S. Application Data
(60) Provisional application No. 61/481,915, filed on May 3, 2011.
(60) Provisional application No. 61/508,181, filed on Jul. 15, 2011.
(60) Provisional application No. 61/529,477, filed on Aug. 31, 2011.
(60) Provisional application No. 61/549,891, filed on Oct. 21, 2011.

(51) Int. Cl. A61B 5/0402; A61B 5/11; A61B 5/00

(52) U.S. Cl. CPC A61B 5/0402; A61B 5/6823; A61B 5/6826; A61B 5/1107
USPC 600/484; 600/527

(57) ABSTRACT

A method and apparatus for assessment of cardiac contractility in a subject by recording precordial acceleration signals. This includes, but is not limited to, the method and apparatus of seismocardiography (SCG).

### FIELD OF THE INVENTION

The present technology pertains in general to technology for assessment of cardiac contractility in a subject by recording precordial acceleration signals. This includes, but is not limited to, the method and apparatus of seismocardiography (SCG). This has applications in cardiac resynchronization therapy (CRT) and monitoring of any cardiac abnormality associated with significant changes of contractility such as ischemic heart disease.

### BACKGROUND

Myocardial contractility is the intrinsic ability of the heart to contract and represents the capability of the heart to produce the required force needed for circulation of blood in the body. There are numerous types of cardiac disorders and abnormalities that decrease myocardial contractility, some examples include ischemic heart diseases, congestive heart failure and myocardial infarction. If adverse changes in cardiac contractility are not diagnosed, monitored and appropriately treated, it will progressively decrease the heart's ability to supply sufficient oxygen to body and can be life-threatening.

Clinical indices of myocardial contractility can be categorized as follows (Arnold Physiology of the heart): based on pressure measurements (such as dP/dtₘₐₓ), volume and dimension (such as stroke volume and ejection fraction) and systolic time intervals (such as pre-ejection period, left ventricular ejection time and isovolumic contraction time). dP/dtₘₐₓ is the gold standard of measurement of myocardial contractility.

Cardiac disorders that result in abnormal stroke volume, and a clinical need to estimate stroke volume non-invasively, can be simplified into four categories 1) filling volume changes, which result from either altered filling pressure, or altered diastolic compliance, (examples: hypovolemic shock from bleeding; increased blood volume from chronic heart or kidney failure) 2) altered effective length of contractile shortening, (example: myocardial infarction with a segment of the myocardium not contracting, as in segmental hypokinesis) 3) altered effective speed of contractile shortening (contractility—example of decreased contractility: generalised cardiomyopathy from many possible causes; example of increased contractility: high adrenalin state in an acute anxiety attack), and 4) altered arterial impedance (example of low impedance: anaphylactic shock or septic shock; example of high impedance: hypertension, atherosclerosis, or adrenergic physiologic response to low cardiac output of any cause).

Clinical trials have demonstrated that CRT results in improved clinical status and lower mortality in selected patients. However, approximately one third of CRT patients fail to respond to CRT due to the inability to 1) identify responders prior to treatment, 2) optimize coronary sinus lead placement during the procedure, and, 3) optimize the interventricular (V-V) and atrioventricular (A-V) interval during surgery. Studies have shown that measurements of left ventricular pressure (LVP) for computation of dP/dtₘₐₓ, which is defined as the maximum value of the first time-derivative of LVP, can be used to optimize lead placement and V-V interval during CRT, increasing the number of responders and positive outcomes reported in patients. However, the measurement of LVP for assessment of dP/dtₘₐₓ is invasive, costly and time-consuming.

Echocardiography is the current, non-invasive assessment choice for evaluating potential responders to CRT, CRT implant optimization and CRT patient outcome. However, even though it has been proposed as a surrogate for dP/dtₘₐₓ, it is not recommended due to its poor reproducibility. Furthermore, echocardiography requires expensive equipment, a skilled operator/technologist to capture the image and an Echocardiologist to interpret the image. For these reasons, clinicians often limit its application in patient selection and monitoring post implant only. Accordingly, other means of non-invasive assessment are being explored.

Every heart beat sets the body into mechanical vibrations that can be recorded using different apparatuses and techniques. Over the past century, extensive research has been conducted on interpretation of these signals in terms of their relationship to cardiovascular dynamics. These apparatuses can be divided into two distinct categories, based on the approach they take to look at the cardiovascular system.

The first category utilizes signals that are created by changes of the centre of mass of the whole or upper part of body as the results of blood circulation. There have been efforts in the past to estimate stroke volume and cardiac output from center of mass recording signals, such as ballistocardiogram (Starr and Noordergraaf, Ballistocardiography in Cardiovascular Research, Lippincott, 1967; Etemadi et al. Conf Proc IEEE Eng Med Biol Soc. 2009;2009:6773-6776).

The second category utilizes measurements made from regions localized near the heart where pulsations over the heart (precordium) are recorded (Weissler, Noninvasive Cardiology, Grune & Stratton, 1974). Seismocardiogram (SCG), apexcardiogram (ACG), pressocardiogram, sternal acceleration ballistocardiography (SAB), kinetocardiogram (KCG), left parasternal cardiogram (LPC) and precordial ballistocardiogram belong to this category. In every heartbeat, because of shape and positional changes of the heart and intracardiac events, the pericardium is vibrated. These vibrations are divided into two different frequency ranges: high frequencies (20-2000 Hz, sonic range) and low frequencies (0-20 Hz, infrasonic range).

High frequency range signals are those produced by intracardiac events such as the opening and closure of the heart valves, ejection, and murmurs and are studied in phonocardiography. Low frequency signals are those produced by shape changes and movements of the heart, during ejection and filling. These two frequency categories of precordial signals may overlap at times in the way they relate to intracardiac events.

Seismocardiography (SCG) is a method of graphically recording minute mechanical movements on an individual's body as a consequence of forces associated with cardiac function, e.g., myocardial contractions and related subsequent opening and closure of valves in the heart. These minute movements are amplified and translated by a pick-up device (e.g., an accelerometer) placed on patient's torso, into signals with electrical potentials in both the infrasonic (less than 20 Hz) and audible (more than 20 Hz or phonocardiography) range.

The rhythmic contractions of the heart under resting and stressed conditions produce repeating SCG wave patterns that enable visual detection and assessment by qualified diagnosticians of normal and abnormal cardiovascular function. SCG manifest the force of cardiac ejection and the timings of cardiac events. As an example, SCG provides a practical means of studying the mechanical response of the heart in its adjustment to the stress of exercise.

Baevskiy and colleagues developed seismocardiography (SCG) in 1964 (Kardiologila 18:87-89). The technique consisted of an accelerometer attached over the left side of the rib cage, which recorded compression waves transmitted through the chest wall from heart contractions. Over the years, SCG was refined as a technique for left ventricular monitoring during ischemia (Salerno and Zanetti Chest; 1991; 100(4):991-993). However, SCG devices were not implemented on a large scale due to the emergence and sudden interest in echocardiography. At present, modern accelerometer-based technology is revitalizing the science of SCG, allowing the motion of the heart to be recorded and analyzed quickly and efficiently for the assessment of cardiac function.

WO2008/095318 describes a system for monitoring and detecting abnormalities in an individual's physiological condition by concurrently detecting and processing an electrocardiograph (ECG) signal and SCG signal. Each signal is analyzed to detect repeating cyclical patterns and characterized to identify individual components of the repeating cycles. At least one component in one signal is selected as a reference marker for a selected component in the other signal and the two signals are then synchronized and output signals is produced.

WO2009/073982 describes a method and apparatus for locating and marking points on a waveform, which includes providing data corresponding to electrocardiogram and seismocardiogram waveforms correlated in time, searching the data to locate points corresponding to cardiac events, a location of each of the points corresponding to cardiac events being defined by a rule set, identifying and storing the points corresponding to cardiac events and outputting a visual representation including the points corresponding to cardiac events marked on the electrocardiogram and seismocardiogram waveforms.

U.S. Pat. No. 6,978,184 describes a method and system for determining the effectiveness of cardiac resynchronization therapy while stimulating a patient's heart at different locations during an electrophysiology study that includes collecting seismocardiographic (SCG) data corresponding to heart motion during paced and un-paced beats of said patient's heart and determining hemodynamic and electrophysiological parameters based on the SCG data.

Marcus et al. (Pacing Clin Electrophysiol. 2007; 30(12):1476-1481) describes accelerometer-derived time intervals during various pacing modes in patients with biventricular pacemakers and compares these with normal subjects.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a method and apparatus for estimating myocardial contractility using precordial vibration signals. In accordance with an aspect of the present invention there is provided a method of assessment of cardiac contractility comprising: processing raw seismocardiogram (SCG) data optionally with ECG data to extract a respiration signal; following identification and extraction of the respiration signal, the signal is passed through band-pass filters having cut-off frequencies of about 0.5 Hz and 20 Hz to obtain the low frequency component; and in parallel from a high pass filter having cut-off frequency of 20 Hz to obtain the high frequency component; annotating cardiac events on the processed SCG data using deterministic rule set approach or the probabilistic machine learning approach or both; extracting features from magnitudes, slope, timing, power and frequency data; and estimating a cardiac contractility index based on said extracted feature using either a patient specific approach or a general regression based approach; wherein optionally in this estimation, different phases of respiration, extracted from the signal previously is considered as an input.

In certain embodiments, the method comprises obtaining seismocardiogram data from said subject. The seismocardiogram data may be obtained using a tri-axial accelerometer or multiple tri-axial accelerometers placed on different points of torso.

In certain embodiments, the contractility index is selected from dP/dt, dP/dtₘₐₓ, dP/dtₘᵢₙ, LV pressure, maximum velocity, acceleration of blood in aorta, stroke volume, ejection fraction, cardiac output and systolic time intervals.

In another aspect of the present invention, there is provided an apparatus for assessing heart contractility in a subject, said apparatus comprising: a sensor device configured to obtain seismocardiogram data indicative of heart motion of the subject measured along one or more spatial axes; optionally a sensor device configured to obtain ECG data; and a computing device communicatively coupled to the sensor device(s) and configured to receive the data therefrom, the computing device configured to: (i) process raw seismocardiogram (SCG) data optionally with ECG data to extract a respiration signal; (ii) process SCG data by passing signal through band-pass filters having cut-off frequencies of about 0.5 Hz and 20 Hz; and in parallel from a high pass filter having cut-off frequency of 20 Hz to obtain the high frequency component; (iii) annotate cardiac events on the pre-processed SCG data; (iv) extract features selected from magnitudes, slope, timing, power and frequency; and (v) estimate an cardiac contractility index based on said extracted features.

In another aspect of the present invention there is provided an apparatus for assessing heart contractility in a subject, said apparatus comprising: a sensor device configured to obtain seismocardiogram data indicative of heart motion of the subject measured along one or more spatial axes; optionally a sensor device configured to obtain ECG data; and a computing device communicatively coupled to the sensor device(s) and configured to receive the data therefrom, the computing device configured to perform the method of the present invention.

### BRIEF DESCRIPTION OF THE FIGURES

These and other features of the technology will become more apparent in the following detailed description in which reference is made to the appended drawings.

FIG. 1 shows the placement of SCG sensor on the sternum for simultaneous recording of precordial acceleration signals and electrocardiogram signal.

FIG. 2 illustrates the proposed methodology for determining an indicator of cardiac contractility from seismocardiogram signal.

FIG. 3 illustrates another apparatus for determining an indicator of a maximum time-rate of change in left ventricular pressure of a subject, in accordance with embodiments of the present technology.

FIG. 4 graphically illustrates obtained electrocardiograph and seismocardiogram for processing in accordance with embodiments of the present technology. MVC=Mitral valve closure; AVO=Aortic valve opening; AVC=Aortic valve closure; MVO=Mitral valve opening.

FIG. 5 graphically illustrates a relationship between electrocardiograph data, seismocardiogram, pressure data, and maximum time-rate of change in left ventricular pressure of a subject, in accordance with embodiments of the present technology. MVC=Mitral valve closure; AVO=Aortic valve opening.

FIG. 6 graphically illustrates functions related to processing of obtained seismocardiogram, in accordance with embodiments of the present technology. MVC=Mitral valve closure; AVO=Aortic valve opening.

FIG. 7 illustrates the device for measurement of SCG. Left: sensor and transceiver; Right: seismocardiogram sensor axes from the perspective of the observer; x—from right to left, y—from head to toe, z—from back to chest.

FIG. 8 illustrates the simultaneous seismocardiogram, phonocardiogram and electrocardiogram signals. The seismocardiogram is high and low-pass filtered to show how these filtered signals complement each other. The lower frequency component is annotated as follows: MVC=mitral valve closure; AVO=aortic valve opening; IM=isovolumic movement; AVC=aortic valve closure; MVO=mitral valve opening; RE=rapid systolic ejection; MA=maximum acceleration of blood in aorta.

FIG. 9 illustrates a cycle of Pig data together with dP/dt signal and ECG (left) and annotated human SCG; MC: Mitral valve closure, IM: isovolumetric moment, AO: aortic valve closure, MA: maximum acceleration of blood in aorta RE: Rapid systolic ejection point (right).

FIG. 10 illustrates the lower body negative pressure setup.

FIG. 11 illustrates dP/dtₘₐₓ for over 600 heartbeats of one of the pigs (bottom) and the time period between the R wave of ECG and the peak of SCG (R-AO) (top).

FIG. 12 illustrates the dP/dtₘₐₓ plotted versus the selected feature (R-AO) for all 30 sessions of the three pigs together.

FIG. 13 illustrates the stroke volume (bottom trace) and RMS of the SCG signal over more than 900 heartbeats of a subject (top) and the stroke volume plotted versus the RMS value (bottom).

### DETAILED DESCRIPTION OF THE INVENTION

The present invention provides a method and apparatus for assessing heart function in a subject by determining an indicator of myocardial contractility such as, dP/dtₘₐₓ+ and dP/dtₘₐₓ−, stroke volume, cardiac output, ejection fraction, left ventricular end systolic volume, left ventricular end diastolic volume and other blood volumes for a subject via precordial accelerograms and vibrograms, such as seismocardiogram (SCG). This assessment may further include assessment of cardiac function with one or more other methods of cardiac monitoring, such as ECG, direct pressure monitoring, echocardiogram, impedance cardiogram, bioreactance and/or heart sounds.

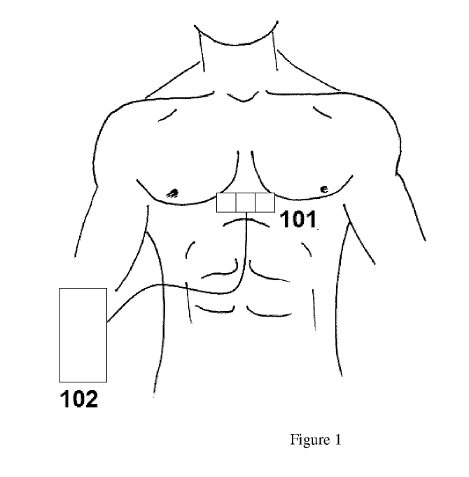

In accordance with the present technology, the method and apparatus utilise seismocardiogram data that is indicative of heart motion measured from the chest along one or more spatial axes, as shown in FIG. 1. In certain embodiments, the seismocardiogram comprises data indicative of heart motion measured along the transverse (or “x”) anatomical axis and/or the anteroposterior (or “z” axis). In certain embodiments, the seismocardsiogram data comprises data indicative of heart motion along the “y” axis.

The methods and apparatus of the present technology may be useful for the detection of potential abnormalities and malfunctions of the cardiovascular system. Any cardiac abnormality that can modify myocardial contractility can manifest itself in morphological changes of SCG; conditions include, but are not limited to, hypovolemic shock from bleeding; increased blood volume from chronic heart failure (systolic and diastolic heart failure) or kidney failure, hypertension, atherosclerosis, cardiomyopathy, myocardial infarct, adrenergic physiologic response or in oncology patients taking cardiotoxic drugs.

The methods and apparatus of the present technology are useful in assessing cardiac function. For example, they may be useful in assessing pulmonary respiratory analysis, global cardiac function, assessing left ventricular function and/or right ventricular function. The methods and apparatus of the present technology may also be useful in assessing patients about to or undergoing cardiac resynchronization therapy (CRT) and/or optimizing CRT. Ongoing monitoring of the heart function of CRT patients can allow, for example, assessment of the effectiveness of the therapy (both short and long term) and/or adjustments to the therapy to improve the patient's status.

### DEFINITIONS

Unless defined otherwise, all technical and scientific terms used herein have the same meaning as commonly understood by one of ordinary skill in the art to which this invention belongs.

There are two categories of mechanical signals created by the heart as mentioned in the Background section. The first category is created because of the movement of central gravity of body with every heartbeat (e.g. Ballistocardiography). In the second category, the local pulsation of the torso is recorded using different techniques (e.g. Apexcardiography, Pressocardiography, etc.). Seismocardiography belongs to this category by recording acceleration of the torso caused by the heartbeat.

As used herein, the term “seismocardiogram” refers to a data obtained from a device for detecting motion, such as vibrations and/or accelerations, due to heart's contraction. For example, the device may comprise one or more motion sensors such as accelerometers for directly or indirectly detecting vibrations or accelerations through the chest wall or other internal or external body area of a subject, said vibrations or accelerations correlated with motion of the heart, blood pumped by the heart, and/or motion of the chest cavity or wall. Monitored vibrations and/or accelerations may correspond to compressive motion due to heart operation, shear motion, or the like, or a combination thereof.

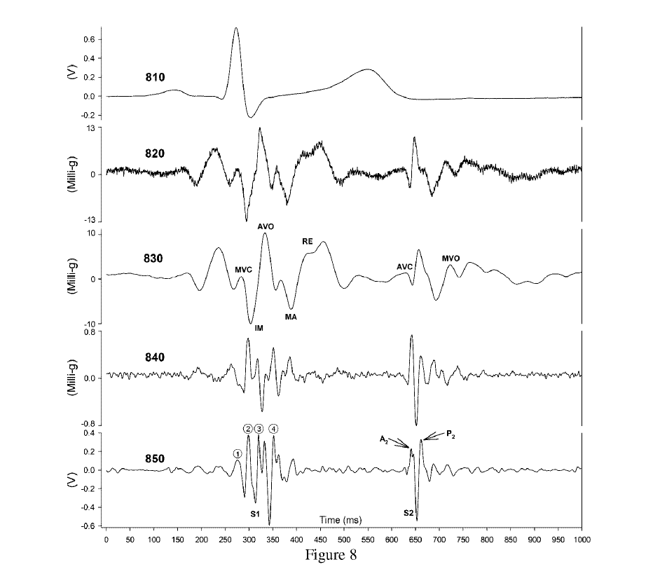

As used herein, the term “magnitude data” refers to data comprising features obtained from direct reading of values from SCG or those obtained from subtracting certain ones from each other. An example of the first type is the value of SCG at the point of AVO (FIG. 8) and an example of the second type is the value of SCG at AVO minus the value of SCG at IM, which corresponds to the peak to peak value of SCG during isovolumic contraction period. The magnitude data can be computed via a norm operation, such as a Euclidean norm operation over data of different axis. Magnitude data comprising accelerometer values measured in plural directions may be combined by vector addition or other means to provide an indication of both magnitude and direction of acceleration, from which magnitude may be extracted. As another example, magnitude data comprising both positive and negative values may be converted to magnitude data comprising positive values only by taking the absolute value of the magnitude data. Area under the curve is also considered as part of the magnitude data category.

As used herein, the term “slope data”, corresponds to those features obtained by calculating the first derivative of signal in certain periods of the cardiac cycle. Such features can include the maximum or average of slope in those periods or the slope calculated at a certain point in time. As an example the slope from IM point to AVO point (FIG. 8).

As used herein, the term “timings data”, when associated with an event, indicates a time at which the event occurred relative to at least one other event or relative to an absolute time scale. The time reference can come from annotation of the simultaneously recorded ECG signal's Q or R wave. Isovolumic contraction time (IVCT or the MVC to AVO period), isovolumic relaxation times (IVRT or AVC to MVO period), pre-ejection period (PEP or Q to AO period), electomechanical delay (EMD or Q to MC period), left ventricular ejection time (LVET or AVO to AVC period), as seen in FIG. 8, are examples of such time features and are known to be related to contractility (Weissler et al. Circulation. 1968; 37(2):149; U.S. Pat. No. 6,978,184).

As used herein, the term “power data” refers to data which consists of those SCG features obtained by calculation of the power or the root mean square (rms) of signal during certain periods of cardiac cycle, as defined in timing data. For example the rms during isovolumic contraction time or pre-ejection period (PEP).

As used herein, the term “frequency data” refers to data which consists of SCG features extracted from power spectral density or wavelet transform of signal in certain periods.

### General Methodology

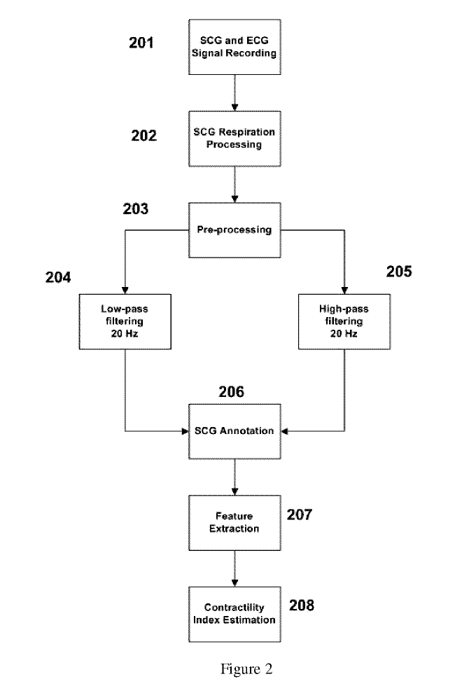

In accordance with an aspect of the present technology, there is provided a method for determining indexes of cardiac contractility. The method comprises processing obtained seismocardiogram in accordance with a predetermined manner. In certain embodiments, the method comprises processing the obtained seismocardiogram in accordance with the predetermined manner set forth in FIG. 2.

SCG signal may be recorded simultaneously with ECG signal 201 as explained in International Patent Application No. PCT/CA2008/000274 (WO2008/095318). Seismocardiogram may be obtained via a sensor device comprising a single-axis or multi-axis accelerometer, configured to measure acceleration due to heart motion along one or more directions necessary to obtain the desired seismocardiogram. The sensor device may be placed external to the subject or at least partially internal to the subject. The sensor device might record acceleration from different points of the torso simultaneously. The length of the data recording may be a short interval under 60 seconds (for example 30 seconds) or longer intervals such as continuous monitoring for several minutes to hours or days. The test may run for any length of time depending on clinician choice. The data might be recorded together with a reference method of measurement of contractility index in a patient specific approach.

The acquired raw SCG signal may be used to extract respiration signal 202. This extraction may also use, the simultaneously recorded, ECG signal to improve the accuracy of respiration extraction as in ECG derived respiration techniques. This respiration signal is used for identification of inspiration and expiration phases. Identification of respiratory phase helps in averaging the SCG signal differently for different respiration phases (Tavakolian et al. Physiol Meas. 2008; 29(7):771-781). The extracted respiration signal serves two purposes. Firstly, to enable a differential analysis of seismocardiogram based on respiration phases. Secondly, to serve as an input to the algorithm (i.e. an adaptive filter) to remove baseline wander of the signal.

After the respiration component is identified and extracted, the raw signal may be high-pass filtered over 0.5 Hz to remove the baseband changes of the signal and smooth the signal 203. This filtering stands for the preprocessing stage. Removal of low frequency level shifts of the signal may also be done through linear piecewise fitting. The preprocessing stage may also include the removal of motion artifacts using manual or adaptive filtering methods using the respiration signal obtained in the previous steps as a reference.

The pre-processed signal obtained from the previous stage may be low pass filtered, under 20 Hz, to obtain the infrasonic, sub-audible, component 204 and also high pass filtered, more than 20 Hz, to obtain the phonocardiogram component 205. These two neighboring frequency bands provide complementary information about valvular opening and closure. FIG. 8 illustrates a sample of such filtering and 820 is the seismocardiogram picked up by the accelerometer from the sternum, in the back to front direction (z). The low-pass filtered signal (i.e. infrasonic component) 830 may be annotated based on the events of the cardiac cycle. In certain embodiments, the low-pass filtered signal 830 is used alone to obtain an assessment of mitral valve closure (MVC) and (AVC).

The high-pass filtered signal (i.e. high frequesncy component) 840 has a very close resemblance to the phonocardiogram signal 850 recorded simultaneously. In certain embodiments because of motion artifacts and/or differences of morphology in different people, it is not feasible to obtain a correct assessment of mitral valve closure and aortic valve closure (MVC and AVC) just from the infrasonic component 830 alone. In these embodiments, the S1 and S2 waves of the high frequency component 840 is used to assist in identification of mitral and aortic valve closure times by narrowing down the search window. The MVC of the infrasonic component 830 occurs very close to the second wave of S1 on phonocardiogram 850 and the AVC occurs very close to A2 wave of S2.

Annotation may relate to associating labels with one or more portions of relevant accelerometer data or data derived at least in part therefrom. In seismocardigraphy terminology, this means the identification of the location of predetermined events in a cardiac cycle (“cardiac events”), such as mitral valve closure (MVC), aortic valve opening (AVO), depolarization of the inter-ventricular septum (Q), isovolumic movement (IM), rapid ejection period (RE), aortic valve close event (AVC), mitral valve open event (MVO) and maximum acceleration of blood in aorta (MA). These cardiac events are identified and annotated automatically, semi-automatically or manually by operator input as in 830 of FIG. 8. The automatic annotation can be done using either deterministic or probabilistic algorithms.

Deterministic annotation of SCG has already been approached before (U.S. Patent Publication No. 2011/0263994; WO 2006/132865). The deterministic approach uses the QRS complex, of the simultaneously recorded electrocardiogram, as reference and follows a rule set to annotate SCG points. Using the higher frequency component of the acceleration signal recorded from the chest help improve the accuracy of these annotations when there is an ambiguity in locating MVC and AVC points. The deterministic rule set approach uses low frequency component in conjunction with high frequency component to fine tune the annotation thereby increasing accuracy.

Probabilistic approach provides a more robust algorithm for annotating SCG signal. As an example of a probabilistic method for annotation of SCG, Hidden Markov Model (HMM) is used, which is a real-time probabilistic method designed for analyzing sequential data (Bernal et al. PLoS Comput Biol. 2007; 3(3):e54). HMM is characterized by a model which has a set of observations—which in this case is the SCG signal—and a generating discrete state sequence model for this set of observations. First the duration of each SCG signal is modelled. This is done by first, annotating many cycles of the SCG signals and to design parametric probability distribution estimation. Since Gamma distribution was successfully used in annotating the ECG signal, it will also be a good candidate for SCG as well but other distributions such as beta distribution would also be suitable. Afterwards, the probability of state transmissions is found by running the training algorithm on the annotated SCG signal. Furthermore, the observation probability may be modelled by either using Gaussian Mixture Model or using time series such as ARMA models. The correct model is the one with the best prediction capability. Such models can be developed on SCG datasets of people with different cardiac abnormality, ages, sexes and races. The accuracy of such algorithm improves by time as more data is fed to it. Hidden Semi-Markov Model (HSMM) is a different implementation of probabilistic modeling and can also be considered.

Based on the annotation of SCG signal 206 different features are extracted from SCG signal 207. Exemplary features are indicative of waveform maximum, minimum or average values during one or more times or ranges of times, maximum, minimum or average slope of a waveform during one or more times or ranges of times, area under a predetermined portion of a waveform, area under the absolute value of a waveform portion, vector direction of multi-dimensional data, other integral or derivative value. These features may be categorized in five groups of magnitudes, slope, timing, power and frequency as explained in the definition section. In a patient specific approach a feature selection methodology may be used to select the SCG features that are more sensitive to myocardial contractility indexes.

The extracted features may be used for estimation and trending of all or some of the contractility indexes. As noted previously, contractility indexes can be categorized: based on pressure measurements (such as dP/dtₘₐₓ), volume and dimension (such as stroke volume and ejection fraction) and systolic time intervals (such as pre-ejection period, left ventricular ejection time and isovolumic contraction time). This trending is done either through a patient specific approach, where for every individual a specific estimator is developed, or through development of general regression equations for different ages, sexes, body mass indexes and chest circumference. In the patient specific approach, an initial determination of the indicator may be used as a “baseline” value and subsequent determinations at later time periods can be compared to this baseline value and any increase or decrease in the value over the baseline can be used as an indication of an increase or decrease in dP/dtₘₐₓ, stroke volume, ejection fraction, cardiac output, left ventricular end systolic volume, left ventricular end diastolic volume and other blood volumes. The method and apparatus in accordance with these embodiments are thus suitable for routine monitoring of subjects in various situations.

### Seismocardiogram Annotation

In certain embodiments, the seismocardiogram is obtained using an accelerometer positioned along a predefined axis and/or having a predetermined orientation. The accelerometer may be a single axis accelerometer or it may be a multi-axis accelerometer, for example a bi-axial or tri-axial accelerometer. The accelerometer may be internally positioned proximal to the heart, for example, for subjects having a pacemaker, the accelerometer may be positioned in or on the housing of the pacemaker. Alternatively, the accelerometer may be externally positioned such that it can detect heart motion through the chest wall as in FIG. 1.

In certain embodiments, the seismocardiogram is obtained by recording from different points on the chest and a high frequency current is also passed through the electrodes resembling impedance cardiography (ICG). In a situation such as these the X and B points of the simultaneous impedance cardiogram can be used for improving the accuracy of detection of AVO and AVC, respectively.

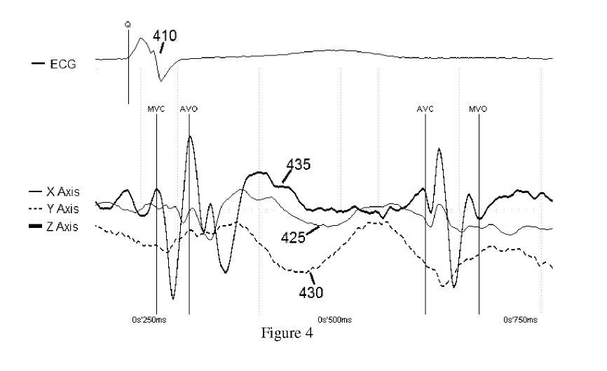

As an initial step, the seismocardiogram can be synchronized with electrocardiograph data obtained from the subject prior to identification of the location of cardiac events. Electrocardiograph data may be used along with seismocardiogram data to identify cardiac events. Specifically, FIG. 4 illustrates a synchronized electrocardiogram waveform 410 and seismocardiogram waveform. The seismocardiogram waveform comprises plural readings from a three-axis accelerometer, identified as follows: the x-axis waveform 425 is shown as a thin, solid line, the y-axis waveform 430 is shown as a broken line confined to the bottom half of the graph, and the z-axis waveform 435 is shown as a thick, solid line having larger peaks than the x-axis waveform. It is noted that the x-axis waveform 425, as shown, represents accelerometer data corresponding to a transverse axis running in the positive direction from subject's right to left, rather than from left to right. A waveform corresponding to a left-to-right transverse axis may be obtained by reflecting the x-axis waveform 425 about the horizontal “zero” axis, as would be readily understood by a worker skilled in the art. Various cardiac events are identified using the reference letters: Q, MVC, AVO, AVC and MVO. The Q annotation denotes depolarization of the inter-ventricular septum; the MVC annotation denotes the mitral valve close event; the AVO annotation denotes the aortic valve open event; the AVC annotation denotes the aortic valve close event and the MVO annotation denotes the mitral valve open event.

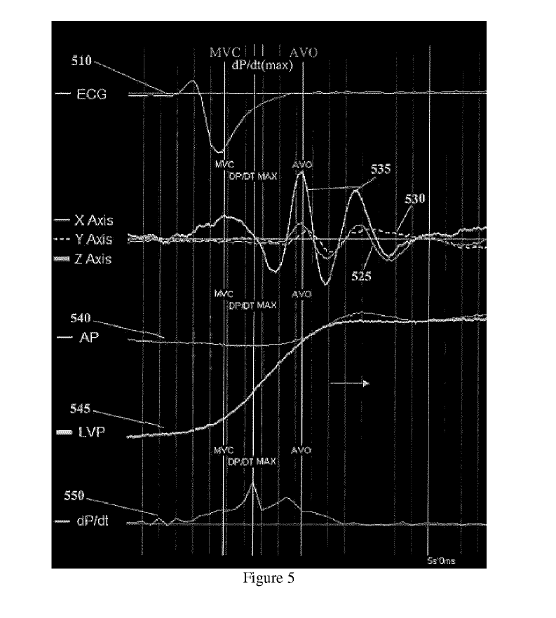

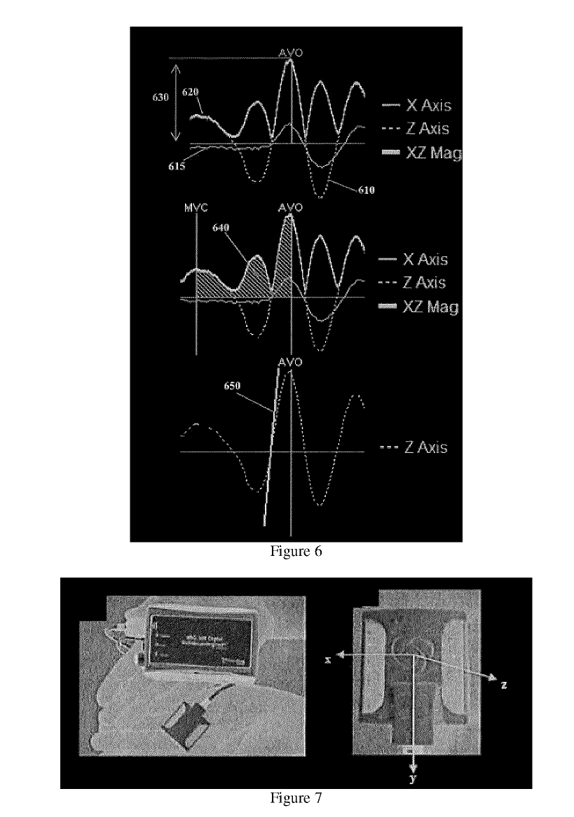

FIG. 5 illustrates an example of a synchronized electrocardiogram waveform 510, an x-axis seismocardiogram waveform 525, a y-axis seismocardiogram waveform 530, a z-axis seismocardiogram waveform 535, an aortic blood pressure waveform 540 (upper, thin), a left ventricular pressure waveform 545 (lower, thick), and a derivative waveform 550 of the left ventricular pressure waveform 545. MVC and AVO events are annotated. dP/dtₘₐₓ is also annotated, and occurs between MVC and AVO.

In some embodiments, one or more predetermined (e.g. cardiac) events are identified via input provided by a trained technician. For example, adequate seismocardiogram may be displayed graphically as one or more waveforms to the technician on a screen, and the technician is instructed to provide input indicative of the time at which the predetermined cardiac events occur. Input may be provided, for example, by moving a vertical line along the time axis of a displayed waveform to a location selected by the technician. In some embodiments, explicit and detailed instructions for identifying the cardiac events may be provided, such that the identification operation is reproducible.

In some embodiments, the seismocardiogram is automatically processed to provide supplementary information for aiding a technician's identification of the one or more predetermined events. Seismocardiogram, electrocardiogram, and/or other relevant physiological data may be processed for this purpose. For example, a time interval containing a set of predetermined cardiac events and substantially excluding other portions of the cardiac cycle may be determined, which may be displayed to the technician. As another example, upon identification of one or more cardiac events by a technician, other related cardiac events and/or time intervals maybe identified. For example, a time interval containing the AVC event may be determined automatically upon identification of the AVO event. On the same note, the higher frequency (more than 20 Hz) component of the signal can be used for rough determination of MVC and AVC points.

Automatic processing may be performed by processing the seismocardiogram to automatically match patterns therein to template patterns stored in memory, the template patterns corresponding to representative, annotated cardiac cycle data. Automatic processing to identify cardiac events may be performed by a system configured to perform pattern matching, such as an expert system, neural network, or other data analysis system as would be readily understood by a worker skilled in the art. Peaks may be identified by comparing adjacent points of time series data, optionally suitably filtered or smoothed, and identifying times at which the data points change from a pattern of increasing with time to a pattern of decreasing with time.

In some embodiments, seismocardiogram is obtained by averaging data, for example taking a mean, median or quantized mode, from plural cardiac cycles, for example to reduce noise while maintaining fidelity of the data. This averaging can be done over inspiration and expiration cycles separately. It is known that cardiac contractility differs between inspiration and expiration and an averaging that considers such difference can greatly assist in interpretation of results. The respiration signal can be derived from the precordial acceleration signals as in 201.

A multi-axis accelerometer may comprise plural accelerometers, each configured for generating accelerometer readings for a different axis. In some embodiments, the plural accelerometers may be substantially co-located. In some embodiments, different accelerometers may be differently located. For example, each accelerometer may be placed so that, as much as is feasible, it generates readings due to compression waves, rather than shear waves. Such configurations may provide an improved set of measurements in some embodiments. Certain cardiac events manifest themselves better on specific points of the thorax. A multiple position recording of seismocardiogram will provide an opportunity to have a more global view of the mechanical performance of the heart similar to multichannel ECG providing a better view of electrical performance of the heart. In an extreme case an acceleration mapping of the chest can be considered by placing an array of equally spaced accelerometers on the chest. Thus, there are also different positions on the chest where the accelerometer can be placed such as sternum, between the ribs, clavicle and suprasternal notch, other arbitrary points on the torso or on all of them simultaneously.

In some embodiments, in which the seismocardiogram comprises data indicative of heart motion measured along at least the z-axis, the seismocardiogram may include an identified MVC event. MVC may be located near a positive peak of the z-axis seismocardiogram immediately prior to a relatively large-amplitude negative peak followed by a relatively large-amplitude positive peak. MVC may be located after said positive peak, for example at or near a point in time where the rate of decrease in the z-axis data becomes substantially constant. In any case MVC happens very close to the beginning of the 51 sound of phonocardiogram 850. High pass filtering of the signal recorded from the chest more than 20 Hz yields a signal 840 which is highly correlated with phonocardiogram. Detection of 51 sound of the high frequency component can assist in detecting MVC as in FIG. 8.

In some embodiment of this invention mitral valve opening point (MVO) can be assesses by an accelerometer or pressure transducer on the point of maximum impulse, similar to apexcardiography. The same setup can also be used for proper assessment of MVC from AVO.

In some embodiments, in which the seismocardiogram comprises data indicative of heart motion measured along at least the z-axis, the seismocardiogram may include an identified MI event and an identified AVO event, in which AVO may be defined as the first positive peak on the z-axis seismocardiogram following MI.

In some embodiments, in which the seismocardiogram comprises data indicative of heart motion measured along at least the z-axis, the seismocardiogram may include an identified MA event. This corresponds to I wave of ballistocardiogram and is very close to the moment when the blood has its maximum acceleration in the ascending aorta.

In some embodiments, in which the seismocardiogram comprises data indicative of heart motion measured along at least the z-axis, the seismocardiogram may include an identified aortic valve closing (AVC) event, which may be defined as the ‘hook’ or ‘knee’ following the T wave location on the ECG, but prior to a large positive peak in the z-axis BCG data. In some embodiments, in which the seismocardiogram comprises data indicative of heart motion measured along at least the z-axis, the seismocardiogram may include an identified mitral valve opening (MVO) and an identified AVC event, in which the MVO may be defined as the first trough on in the z-axis BCG data after the z-axis peak, which is at least 50 ms after AVC. The high frequency (more than 20 Hz) component of the signal recorded from the chest 840 has a very close correspondence to phonocardiogram 850 and the first wave of S2 (A2) corresponds to the closure of aortic valve thus, detection of S2 from the high-frequency component of the signal assists in proper detection of AVC.

The above methods for locating cardiac events are exemplary. Other definitions and/or methods for locating cardiac events such as MVC and AVO may be used, as would be readily understood by a worker skilled in the art.

In embodiments of the present technology, processing, annotation, or both may be performed manually, automatically, or semi-automatically. Manual processing, annotation, or both may comprise one or more steps performed by a technician, for example following a predefined rule set, in which one or more annotations are derived in a predetermined manner from a provided data set. Automatic processing, annotation, or both may comprise one or more steps performed by a computer, for example following a predefined algorithm, in which one or more annotations are derived and displayed in a predetermined manner from a provided data set. Semi-automatic processing, annotation, or both may comprise one or more computer-performed steps, with certain input provided by a technician in an interactive manner.

### Apparatus

Embodiments of the present technology provide an apparatus for assessing heart contractility in a subject. The apparatus is configured to determine an indicator of cardiac function by obtaining and processing seismocardiogram. The apparatus comprises one or more sensor device(s) and a computing device in communicative and/or operative cooperation. The sensor device is configured to obtain seismocardiogram indicative of heart motion of the subject measured along one or more spatial axes. The sensor device may comprise one or more internal or external accelerometers or other adequate sensors. The computing device is configured to receive the seismocardiogram and process at least a portion of same in accordance with a predetermined function.

The sensor device comprised by the apparatus may comprise a single-axis, double-axis, or multi-axis accelerometer. The sensor device may further be configured to obtain other data, such as electrocardiogram, impedance cardiogram or other data indicative of a subject's physiological condition. In some embodiments, the apparatus may further include additional cardiac monitoring devices. For example, the apparatus may further include an ECG, a device to monitor heart sounds, a blood pressure monitor and combinations thereof.

In some embodiments, the sensor is associated/incorporated into an article, such as a shirt, worn by the patient. Such wearable device allows for recording of data in daily life and can be worn by the patient at any time (both when awake or asleep) and allows for continuous monitoring.

An example of an external sensor device for detecting both ECG and seismocardiogram signals is described in International Application No. PCT/CA2008/002201 (WO2009/073982), which is herein expressly incorporated by reference in its entirety. Another example of a sensor device is a transoesophageal sensor such as that described in International Patent Application No. PCT/CA2009/00111 (Publication No. WO2010/015091), herein expressly incorporated by reference in its entirety. Another example of a sensor device for detecting BCG signals is a dBG300™ sensor provided by Heart Force Medical Inc, which generates signals indicative of forces due to heart motion via a tri-axial accelerometer and transmits said signals for processing via a computer such as a laptop. In one embodiment of the present technology, the apparatus comprises an external sensor device.

Examples of sensor devices comprising an accelerometer that are configured for internal placement are known in the art and include, for example, a micromass uniaxial acceleration sensor manufactured by Sorin Biomedica Cardio SpA (Saluggia, Italy), which is configured for placement in the pacing lead of a pacemaker device. Other types of accelerometers known in the art could be utilized for placement in or on the pacemaker housing.

An accelerometer may be configured to detect and output a signal indicative of motion, such as magnitude and direction of acceleration, and may be a piezoelectric, piezoresistive, capacitive, MEMS or other type of accelerometer.

The apparatus further generally comprises a general or special purpose computer operatively coupled to the sensor device. The computer may be configured to perform analysis, for example via appropriate hardware, software, firmware, or a combination thereof. A computing device may comprise one or more microprocessors operatively coupled to memory and configured to perform numerical processing operations as would be readily understood by a worker skilled in the art. The memory may contain instructions for performing the processing operations and may also store data for processing and/or data resulting from processing. The computer may be hand-held or may be in the form of a desktop or laptop computer, for example running Windows™ or another operating system along with data acquisition and processing software configured in accordance with the present technology. Data acquisition and processing may be enabled by proprietary software or by software written in C, C++, Fortran, or on a commercially available platform such as LabVIEW™ or LabWindows™/CVI. Processing of acquired data may alternatively be performed by a spreadsheet program or software suite such as MATLAB™. The computer comprises at least a wired or wireless communication port configured to communicate with the sensor device via a standard protocol such as USB™ or Bluetooth™, or via other protocols.

In some embodiments, the apparatus comprises a sensor device and a computing device in a substantially integrated package. In some embodiments, sensor devices and/or computing devices may be separate but coupled via wired or wireless communication.

FIG. 1 illustrates an apparatus in accordance with embodiments of the present technology. The apparatus includes a sensor device 101 for coupling to a subject and a computing device 102 that is in communication with the sensor device. The communication may be wired or wireless. The sensor device is provided for detecting, converting and transmitting digital signals corresponding to seismocardiogram signals. In some embodiments, as shown in FIG. 1, the sensor device 101 is placed on the sternum of the subject for sensing movement of the chest wall. The computing device 102 is provided for receiving the digital signals from the sensor device 101 and analyzing the digital signals. The computing device 102 includes a radio device (not shown), a user interface (not shown), a processor (not shown) and a computer memory (not shown) that stores software that is executable by the processor. The software may alternatively be stored on another type of computer readable medium. The computing device 102 controls the sensor device 101 by sending commands, for example wirelessly via the radio device or by a wired interface, in order to initiate and terminate detection and transmission of the BCG signals.

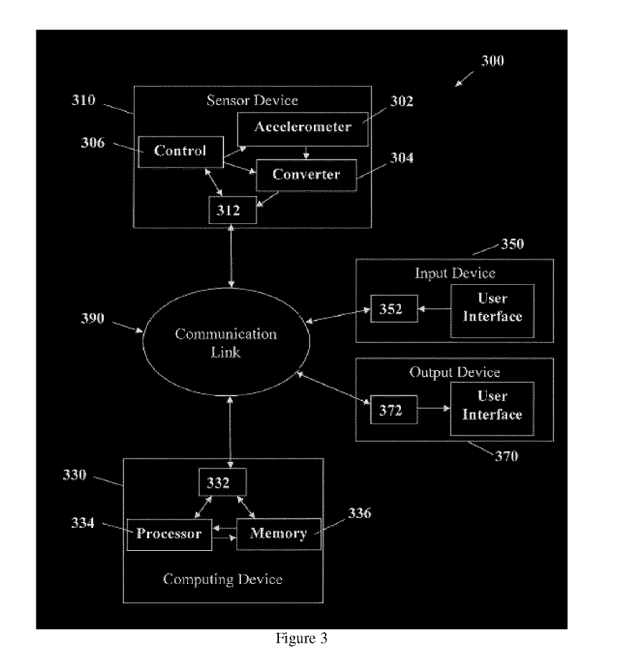

FIG. 3 illustrates an apparatus 300 in accordance with embodiments of the present technology. The apparatus 300 comprises a sensor device 310, a computing device 330, and optionally an input device 350 and/or an output device 370. The sensor device 310, computing device 330, input device 350 and output device 370 are communicatively coupled by communication interfaces 312, 332, 352, and 372, respectively, to each other via a communication link 390, such as a wired, wired, direct or networked link. Optionally, the input device 350 and output device 370 may be part of the computing device 330, in which case the communication interfaces 352, and 372, may be omitted. The sensor device 300 comprises an accelerometer 302, such as a three-axis accelerometer, along with an analog-to-digital converter 304 for converting accelerometer readings into digital format for communication via interface 312. A control unit 306 is provided to convey commands such as “start” and “stop” commands, calibration commands, and/or other commands to components of the sensor device 310. The computing device 330 comprises a processor 334 operatively coupled to memory 336. The processor 334 and/or memory 336 receive seismocardiogramfrom the sensor device via interface 312.

### Applications

The methods and apparatus of the present technology are useful in assessing cardiac function. For example, they may be useful in assessing global cardiac function, assessing left ventricular function and right ventricular function together and/or in isolation. In some embodiments, they may be useful for the assessment of left ventricular and right ventricular hemodyanamics alone or in isolation.

In some embodiments, the methods and apparatus of the present technology may also be useful in assessing patients about to or undergoing cardiac resynchronization therapy (CRT) and/or optimizing CRT. For example, they may be useful for the optimization of pacing mode, optimization of delays, optimization of lead placements, identification of potential responders pre-CRT and/or monitoring. Ongoing monitoring of the heart function of CRT patients can allow, for example, assessment of the effectiveness of the therapy (both short and long term) and/or adjustments to the therapy to improve the patient's status.

In some embodiments, there is provided use of the apparatus with single electrode configuration or plurality of electrodes, including quadripolar lead such as those manufactured by St Jude Medical or other Medtronic devices, to optimize CRT.

In some embodiments, the methods and apparatus of the present technology may be useful in prognostic models predicting effectiveness of the BiV pacemakers in patients with coronary heart failure symptoms and conduction abnormalities.

In some embodiments, the methods and apparatus of the present technology may be useful for the detection of potential abnormalities and malfunctions of the cardiovascular system. For example, they may be useful in the assessment of systolic and diastolic heart failure and heart disease or the detection of cardiomyopathy, including but not limited to cardiomyopathy in athletic hearts. This assessment may be useful in the determination of appropriate therapy and ongoing monitoring would allow for an assessment of the effectiveness of the therapy and/or adjustments to the therapy to improve the patient's status.

In some embodiments, the methods and apparatus of the present technology may be useful in the identification of systolic dysfunction in patients with a history of heart failure. In some embodiments, the methods and apparatus of the present technology may be useful in the identification of diastolic dysfunction in patients with a history of heart failure. In some embodiments, the methods and apparatus of the present technology may be useful in assessment and/or prevention of cardiotoxic drugs in patients.

In some embodiments, the methods and apparatus of the present technology may be used to assess hemodynamics in patients at a single time point and/or over time. In some embodiments, the methods and apparatus of the present technology may be useful in the absolute hemodynamic assessment in patients. In some embodiments, the methods and apparatus of the present technology may be useful in relative hemodynamic assessment (changes over time) in patients. In some embodiments, the methods and apparatus of the present technology may be useful in any combination of absolute or relative hemodynamic

The methods and apparatus are also useful in monitoring progression of heart disease in a subject, and/or in monitoring the effect of drugs or other therapies in patients with heart disease or other cardiac conditions. In some embodiments, they are useful in long-term monitoring of systolic dysfunction in patients with a history of heart failure.

The methods and apparatus of the present technology may also be useful in assessing heart function in healthy subjects, for example, to monitor an improvement in heart function as the result of a diet or exercise regimen. The methods and apparatus of the present technology may also be useful in the classification of patients with and without heart failure.

The method and apparatus of the present technology may be utilized, for example, in out-patient clinics, ambulatory clinics, hospitals, doctor's offices, catheter laboratory, emergency department, echocardiography department, oncology, ambulance, at home, in the field (for example, during military or rescue operations), for insurance assessments, sports medicine clinics and sporting venues.

In certain embodiments, the indicator determined by the methods described herein can be a relative indicator of dP/dt, dP/dtₘₐₓ+ and dP/dtₘₐₓ−, stroke volume, cardiac output, ejection fraction, left ventricular end systolic volume, left ventricular end diastolic volume and other blood volumes.

The invention will now be described with reference to specific examples. It will be understood that the following examples are intended to describe embodiments of the invention and are not intended to limit the invention in any way.

### EXAMPLE

### Example 1

### Myocardial Contractility: A Seismocardiography Approach

### Introduction

Myocardial contractility is the intrinsic ability of the heart to contract. Different levels of contractility are assigned by different degrees of binding between myosin and actin filaments. The Gold standard for assessment of myocardial contractility is the invasive measurement of changes of pressure in the ventricle, through the use of catheters, during the cardiac cycle and calculation of the dP/dtₘₐₓ index [1-3]. Contractility is reduced in variety of cardiac abnormalities and it is therefore advantageous to have a non-invasive method for assessment of these reductions. Stroke volume is another index of contractility and a close correlate of dP/dtₘₐₓ. Seismocardiography (SCG) has been proposed in the past for estimation of stroke volume [4-6].

In this example the estimation of cardiac contractility based on SCG was investigated with two separate approaches. In the first, the preliminary results on the association of SCG parameters with dP/dtₘₐₓ are presented. This is the first approach to use SCG for estimation of dP/dtₘₐₓ.

In the second, the association of stroke volume with SCG was evaluated. Unlike previous approaches [5] which used equations for estimating stroke volume, this approach used actual measures of stroke volume recorded simultaneous with SCG and unlike [4] the stroke volume of the subjects were modified over a wide range with the use of lower body negative pressure (LBNP).

### Methodology:

A. SCG Comparison with dP/Dt_{Max}

Ten, female, nonatherosclerotic swine, aged 11.3 to 12.1 weeks with body weights of 29.1 to 38.7 kg were used. This species was chosen as it has been extensively used for studies in the field of cardiology, resulting in a large volume data generated on the cardiovascular response properties and its correlation to human cardiovascular response. The swine and human hearts have correlatively similar anatomy which allows for a more direct human correlation.

All animals were tranquilized and anesthesia was induced intravenously and after intubation maintained by artificial ventilation with oxygen and isoflurane. The animal was placed in dorsal recumbency. Limb-leads were placed for electrocardiographic (ECG) monitoring. Each animal's sternum area was shaved for placement of the dBG® proprietary sensor (Heart Force Medical Inc., Vancouver, Canada). A trained operator applied the sensor on the sternum in the midline, with the lower edge of the sensor placed approximately 3 cm above the xiphoid process the same place as advise by [7] for human studies. All procedures were approved by the Comité Institutionnel de Protection des Animaux d'AccelLAB and complied with the Canadian Council on Animal Care regulations.

After anesthesia induction, the left and right femoral arteries were accessed through an inguinal incision for left ventricular recordings. The right jugular vein was accessed for pacing lead placement. A 7F guiding catheter (Medtronic, Minn., USA) and sensor-tipped PressureWire® (St. Jude Medical Inc., MN, USA) were inserted into the left femoral artery and placed in the apex of the left ventricle for the measurement of left ventricular pressure. Computation of dP/dtₘₐₓ was completed using the RadiAnalyser® Xpress system (St. Jude Medical Inc., MN, USA) and PhysioMon 2.02 software.

The right atrium was paced at 10 separate heart rate (HR) conditions: 90, 100, 110, 120, 130, 140, 150, 160, 170 and 180 bpm for a duration of one minute for each of the heart rates. All HR conditions were counter-balanced to minimize the order effect. Left ventricular pressure, aortic and left atrial pressure, ECG and SCG data collection were synchronized by the use of a Biopac (MP150, Biopac Systems Inc., CA, USA) and sampled at 1000 Hz.

B. SCG and Stroke Volume in Humans During LBNP

An orthostatic stress test of graded lower body negative pressure (LBNP) was used to change central blood volume and thereby reduce cardiac stroke volume. Lower body negative pressure simulates reduction in central blood volume similar to hemorrhage; however, the blood volume is not lost but is instead trans-located to the lower portions of the body. The participant's lower body was placed in a negative pressure chamber and sealed at the iliac crest as in FIG. 10. Vacuum was applied to the chamber to drop the pressure at 10 mmHg decrements, 6-minutes in duration through −60 mmHg. The pressure was increased in the same fashion in 6 minutes to reach normal pressure. The level of −40 mm Hg produces similar volume shifts associated with complete upright posture.

Five young and healthy male participants took part in this study with average age, weight and height of (32.8±6.1 years, 82.2±11.9 kg and 176.6±4.45 cm). The signal recording was performed at Aerospace Physiology laboratory under an ethics approval from Simon Fraser University.

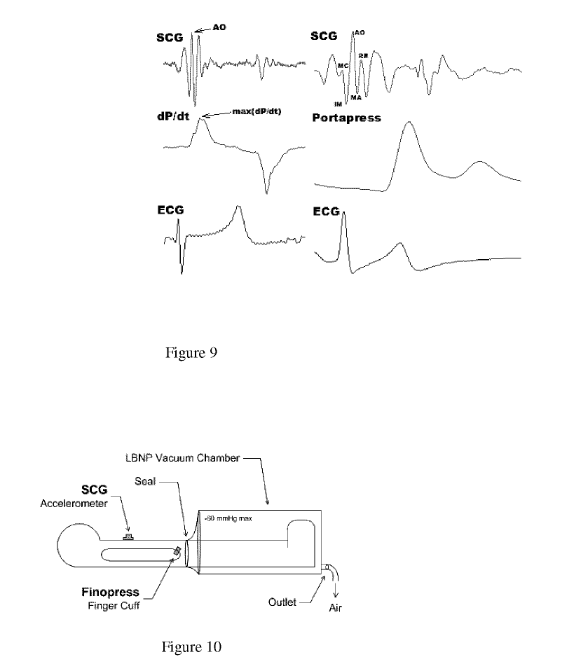

The SCG signal was measured with a high sensitivity accelerometer (1000 milivolts/g, factory calibrated, mass of 54 grams) as used in [4]. The participants were in the supine position and the signals were recorded in back to front direction, perpendicular to the body surface. ECG signal was also acquired and used to segment the cardiac cycles. The stroke volume was measured using Portapres device from Finapres Medical Systems. All signals were recorded using National Instrument DAQs. A snapshot of the recorded signals can be seen in FIG. 9. The signal annotation as proposed in [7] are also shown.

### Results:

A. dP/dtₘₐₓ in Pigs

The R wave of ECG, recorded from the pigs, was used to segment heart beats. Inspired by the annotation of human SCG[7], eighteen morphological features were extracted from every SCG beat, including amplitudes, slopes and timings of peaks and valleys. Three pigs were completely analyzed for this paper.

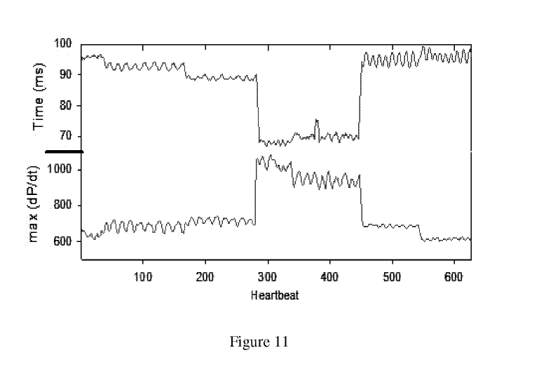

From the simultaneous dP/dt signals dP/dtₘₐₓ was calculated by finding the maximum of the signal in a 200 ms window, after R wave of ECG, as can be seen in FIG. 9. A plot of dP/dtₘₐₓ over all heartbeats of one of the pigs can be seen in FIG. 11 together with one of the feature extracted simultaneously from SCG.

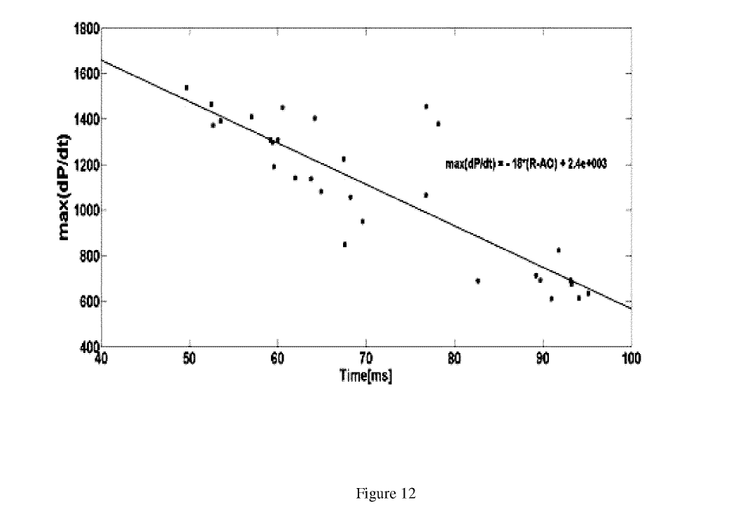

For the three pigs of this study, the average of dP/dtₘₐₓ was calculated over a whole recording session corresponding to every hear rate, and the same averaging was performed for the eighteen SCG-extracted features. A stepwise regression was performed over accumulation of all the features. The time period between the R waves of ECG to the peak of SCG (R-AO) was the selected feature. The correlation coefficient over the data of all three pigs together was −0.86 and is plotted in FIG. 12. These results suggest an association between the dP/dtₘₐₓ and features extracted from SCG of the pigs.

TABLE 1
Correlation coefficients between the
dP/dtₘₐₓ for every pig and the selected SCG feature.

| Pig | 1 | 2 | 3 | Average |
|---|---|---|---|---|
| r | −0.75 | −0.94 | −0.91 | −0.87 |

B. Stroke Volume in Humans

Similar to the pig data, the R-wave of ECG was used to segment heart beats and morphological features were extracted from every SCG beat using software developed in Matlab. There were more than 930 cardiac cycles per subject.

Sixteen features were extracted from the SCG signal in four categories; timing (R-MC, R-AO, R-MI and MC-AO), amplitude (MC, AO, MI, MI-AO), slopes (MI to AO, MC to MI, MA to RE) and root mean squares (RMS1: rms 150 ms after R wave, RMS2: rms during isovolumic contraction period).

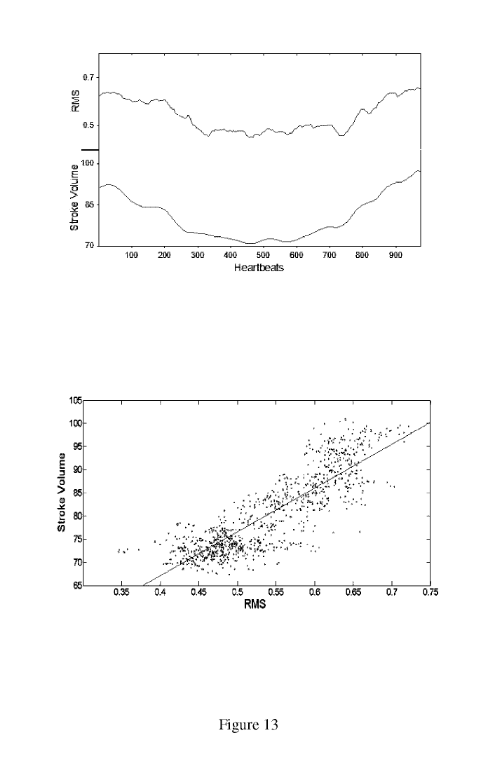

The stroke volume was calculated by the Beatscope software from the Portapres's waveform as in FIG. 9. A plot of the stroke volume for one of the subjects can be seen on top of FIG. 13 together with one of the SCG extracted features (RMS).

The first row of Table 2 shows the R-squared value of a multivariate regression over all sixteen features. For every individual feature the r2 value was calculated and for every subject the three features with maximum r2 are reported in the middle of Table 2. A mixed stepwise regression was performed on the data from each subject and six features common between all of the five subjects (MC, MA, MI-AO slope, MC-MI slope, RMS1 and R-AO) were selected. A multivariate regression which included all six selected variables was then performed for each subject to provide the r2 in the second to last column of Table 2. The selected features are from all four categories and the resulting r2 are quite high. The final column in Table 2 represents the correlation coefficient for the SCG variable R-AO.

### Discussion:

As explained in the second part of previous section, a variety of extracted SCG features were compared in this study with the intent to select the best features for every subject. It was observed that the features extracted from the amplitude of the SCG signal were not as good when a compared to timing features in two of the subjects (subjects one and five). It is obvious that as more features are added the lower the estimation error; nevertheless, if only a few features are to be selected, then timing features may provide the better candidate.

The timing feature that stood out was the period between the R-wave of ECG to the AO point of SCG. This corresponds to Pre-Ejection Period (PEP). The pig data also indicated that a similar feature to this correlated well with dP/dtₘₐₓ. It is also understood from the literature that reduction in stroke volume and contractility increases PEP [8]. This inverse effect was observed with the high negative correlation in both the pig and human data (Tables 1 and 2).

### CONCLUSION

In our previous study we used general regression and nonlinear estimators to predict stroke volume, obtained through Doppler ultrasound [6]. That study presented a patient-specific solution to estimation of stroke volume in which, the algorithm was trained on the data of every individual subject separately. Thus, all possible morphological features were fed to the estimator to increase its accuracy. In this study we were more focused on the different effects of every individual feature on the indexes of contractility and also on a wider range of stroke volume.

As was expected because the inherent nature of SCG and the different ways the hearts are located in rib cages of different people certain features of the mechanical vibration in SCG are more dominant in different people. Although the classical looking SCG proposed in [7] are quite common, different morphologies exists in other people with normal cardiovascular function.

Motion artifacts affect mechanical signals such as SCG significantly, making it very difficult to conduct experiments such as stress tests to modify stroke volume. LBNP on the other hand, provides a stable experimental platform to change the central hemodynamics and study the corresponding SCG changes.

TABLE 2
The r squared values for different subjects and different combination of
features.

| | | Three | | | r² of six | |
|---|---|---|---|---|---|---|
| | r² on all | Selected | r² of Best | | common | r for R-AO |
| Subjects | Features | Feature | Feature | Category | Features | feature |
| 1 | 0.90 | R-MC, R- | 0.69 | Timings | 0.77 | −0.80 |
| | | MI, R-AO | | | | |
| 2 | 0.99 | MC, R- | 0.92 | Amplitude | 0.94 | −0.94 |
| | | AO, R-MI | | and | | |
| | | | | Timings | | |
| 3 | 0.98 | RMS2, | 0.98 | All | 0.96 | −0.92 |
| | | MC, R- | | | | |
| | | AO | | | | |
| 4 | 0.92 | R-MC, R- | 0.81 | Timings | 0.98 | −0.90 |
| | | AO, R-MI | | | | |
| 5 | 0.96 | R-AO, R- | 0.93 | Amplitude | 0.87 | −0.96 |
| | | MC, MC | | and | | |
| | | | | Timings | | |
| Average | 0.95 ± 0.03 | R-AO | 0.87 ± 0.10 | Timings | 0.90 ± 0.07 | −0.90 ± 0.06 |

### REFERENCES

[1] F. Jamal, J. Strotmann, F. Weidemann, T. Kukulski, J. D'hooge, B. Bijnens, F. Van de Werf, I. De Scheerder and G. R. Sutherland. Noninvasive quantification of the contractile reserve of stunned myocardium by ultrasonic strain rate and strain. Circulation 104(9), pp. 1059-1065. 2001.
[2] P. Houthuizen, F. A. Bracke and B. M. van Gelder. Atrioventricular and interventricular delay optimization in cardiac resynchronization therapy: Physiological principles and overview of available methods. Heart Fail. Rev. 16(3), pp. 263-276. 2011.
[3] E. Donal, L. Giorgis, S. Cazeau, C. Leclercq, L. Senhadji, A. Amblard, G. Jauvert, M. Burban, A. Hernandez and P. Mabo. Endocardial acceleration (sonR) vs. ultrasound-derived time intervals in recipients of cardiac resynchronization therapy systems. Europace 13(3), pp. 402-408. 2011.
[4] W. P. McKay, P. H. Gregson, B. W. McKay and J. Militzer. Sternal acceleration ballistocardiography and arterial pressure wave analysis to determine stroke volume. Clin. Invest. Med. 22(1), pp. 4-14. 1999.
[5] P. Castiglioni, A. Faini, G. Parati and M. Di Rienzo. Wearable seismocardiography. Conf. Proc. IEEE Eng. Med. Biol. Soc. 2007 pp. 3954-3957. 2007.
[6] K. Tavakolian, A. Blaber, B. Ngai and B. Kaminska, “Estimation of hemodynamic parameters from seismocardiogram,” in Computing in Cardiology, Belfast, Ireland, 2010, pp. 1055-1058.
[7] D. M. Salerno and J. Zanetti. Seismocardiography for monitoring changes in left ventricular function during ischemia. Chest 100(4), pp. 991-993. 1991.
[8] A. Weissler, Noninvasive Cardiology. New York: Grune & Stratton, 1974.

It is obvious that the foregoing embodiments of the invention are examples and can be varied in many ways. Such present or future variations are not to be regarded as a departure from the spirit and scope of the invention, and all such modifications as would be obvious to one skilled in the art are intended to be included within the scope of the following claims.

1. A method of assessment of cardiac contractility comprising:
processing raw seismocardiogram (SCG) data optionally with electrocardiographic (ECG) data to extract a respiration signal;
following identification and extraction of the respiration signal, passing the respiration signal through band-pass filters having cut-off frequencies of about 0.5 Hz and 20 Hz to obtain a low frequency component and in parallel from a high pass filter having cut-off frequency of 20 HZ to obtain a high frequency component;
annotating cardiac events on the processed SCG data using deterministic rule set approach, a probabilistic machine learning approach, or both;
extracting features from magnitudes, slope, timing, power and frequency data; and
estimating a cardiac contractility index based on the extracted features using either a patient specific approach or a general regression based approach.

2. The method of claim 1, further comprising:
obtaining seismocardiogram data from a subject.

3. The method of claim 2, wherein the seismocardiogram data is obtained using a tri-axial accelerometer or multi tri-axial accelerometer placed on different points of torso.

4. The method of claim 1, wherein the contractility index is selected from dP/dt, dP/dtₘₐₓ, dP/dtₘᵢₙ, left ventricular (LV) pressure, maximum velocity, acceleration of blood in aorta, stroke volume, ejection fraction, cardiac output, and systolic time intervals.

5. An apparatus for assessing heart contractility in a subject, comprising:
a sensor device configured to obtain data indicative of heart motion of the subject measured along one or more spatial axes;
an optional sensor device configured to obtain electrocardiographic (ECG) data; and
a computing device communicatively coupled to the sensor device, the optional sensor device, or both, and configured to receive the data from the sensor device, the optional sensor device, or both, wherein
the computing device is configured to:
process raw seismocardiogram (SCG) data optionally with the ECG data to extract a respiration signal,
process the SCG data by passing a signal through bad-pass filters having cut-off frequencies of about 0.5 Hz and 20 Hz to obtain a low frequency component and in parallel from a high pass filter having cut-off frequency of 20 Hz to obtain a high frequency component,
annotate cardiac events on pre-processed SCG data,
extract features selected from magnitudes, slope, timing, power and frequency, and
estimate cardiac contractility index based on the extracted features.

6. (canceled)

7. The apparatus of claim 5, wherein the computing device is further configured to obtain the seismocardiogram data from the subject.

8. The apparatus of claim 5, wherein the seismocardiogram data is obtained using a tri-axial accelerometer or multi tri-axial accelerometer placed on different points of torso.

9. The method of claim 5, wherein the contractility index is selected from dP/dt, dP/dtₘₐₓ, dP/dtₘᵢₙ, left ventricular (LV) pressure, maximum velocity, acceleration of blood in aorta, stroke volume, ejection fraction, cardiac output, and systolic time intervals.
